(19) United States
(12) Patent Application Publication
(10) Pub. No.: US 2022/0298465 A1
(43) Pub. Date: Sep. 22, 2022

(54) IMPROVEMENTS IN AND RELATING TO THE MONITORING OF CELL EXPANSION

(71) Applicants: Global Life Sciences Solutions Operations UK LTD, Sheffield (GB); The Regents of the University of California, Davis, CA (US)

(72) Inventors: Paul C. Goodwin, Issaquah, WA (US); Paul Anthony Bowles, Toronto (CA); Rohin Iyer, Toronto (CA); Yarden Gratch, Toronto (CA); Mitch M. McCartney, Davis, CA (US); Mei S. Yamaguchi, Davis, CA (US); Cristina E. Davis, Davis, CA (US)

(21) Appl. No.: 17/617,274

(22) PCT Filed: Jun. 9, 2020
(86) PCT No.: PCT/EP2020/065927

§ 371 Date: Dec. 7, 2021

Related U.S. Application Data
(63) Continuation of application No. 16/441,883, filed on Jun. 14, 2019.

(51) Int. Cl. C12M 1/34; C12N 5/075; C12N 5/0783; G01N 27/62; G01N 30/02; G01N 33/00; G01N 33/483

(52) U.S. Cl. CPC C12M 41/36; C12N 5/0609; C12N 5/0636; G01N 27/62; G01N 30/02; G01N 33/0047; G01N 33/4833; G01N 2030/025; G01N 2458/15

(57) ABSTRACT

Disclosed is a method for monitoring cell density during cell expansion resulting from a cell culture process in a bioreactor comprising the steps of: a) cultivating cells in a bioreactor culture chamber according to a cell culture process having cell culture parameters; b) during said process, introducing cell culture fluid inputs and generating waste materials; c) determining the intensity of volatile organic compounds (VOCs) and their chemical species in the waste materials; and d) estimating the density or population of cells in the bioreactor based on said determination.

### FIELD OF THE INVENTION

The present invention relates to apparatus and methods for monitoring of cell expansion, particularly for estimating cell density during cell expansion in a generally closed bioreactor by analysing volatile organic compounds (VOCs).

### BACKGROUND OF THE INVENTION

Some development of the use of VOCs in cellular technologies has been reported, for example:

Within process analytical technologies (PAT), it is known that downstream VOC emissions from cell cultures can be utilized by soft sensors for online bioprocess monitoring. A few examples of this have been demonstrated by measuring cellular VOCs with various technologies. It is known to use a so called ‘electronic nose’ which is a monitor of chemical reactions/binding to show total VOC profiles of Chinese Hamster Ovary (CHO) cells tracked with relation to growth in a bioreactor¹. Biomass and growth rates were predicted from VOC profiles of Escherichia coli batch cultivations², and VOCs were used to detect VOC changes in animal cell reactor cultures due to microbial and viral contaminations, including E. coli.³ However, one major disadvantage of the electronic nose technologies mentioned above is the lack of structural information to confidently identify chemical species, which would be an important step toward assessing the biological relevance of targeted VOCs in any analysis. In addition, those sensors drift over time and must constantly be recalibrated, regenerated or replaced.

Other reports have noted that changes in VOC content in headspace can be measured from mammalian cells using traditional mass spectrometry, and those changes correlated with single gene expression levels.⁴ Mass spectrometry techniques provide additional information for compound identification and have trended towards incorporation as online sensors in reaction monitoring⁵, including bioreactors. Proton transfer reaction-mass spectrometry (PTR-MS) was incorporated into an E. coli bioreactor and VOC profiles correlated to culture growth⁶.
1. Bachinger, T.; Riese, U.; Eriksson, R.; Mandenius, C. F., Monitoring cellular state transitions in a production-scale CHO-cell process using an electronic nose—J Biotechnology and Bioengineering 2000, 76 (1), 61-71. https://www.ncbi.nlm.nih.gov/pubmed/10784297 , discloses sampling the off-gas of biocultures (CHO cells) by means of a ‘nose’ i.e. chemical reactions or molecular binding for detecting changes in cell cultures.
2. Bachinger, T.; Martensson, P.; Mandenius, C. F., Estimation of biomass and specific growth rate in a recombinant Escherichia coli batch cultivation process using a chemical multisensor array—J Biotechnology and Bioengineering 1998, 60 (1-2), 55-66. https://www.ncbi.nlm.nih.gov/pubmed/9571802, discloses a multi-sensor array for bacterial cultivation monitoring.
3. Kreij, K.; Mandenius, C. F.; Clemente, J. J.; Cunha, A. E.; Monteiro, S. M. S.; Carrondo, M. J. T.; Hesse, F.; Molinas, M. D. M. B.; Wagner, R.; Merten, O. W.; Geny-Katinger, C.; Martensson, P.; Bachinger, T.; Mitrovics—On-line detection of microbial contaminations in animal cell reactor cultures using an electronic nose device. J Cytotechnology 2005, 48 (1-3), 41-58. https://www.ncbi.nlm.nih.gov/pmc/articles/PMC3449723/ discloses the use of an electronic nose (EN) device was used to detect microbial and viral contaminations in a variety of animal cell culture systems.
4. Aksenov, A. A.; Gojova, A.; Zhao, W.; Morgan, J. T.; Sankaran, S.; Sandrock, C. E.; Davis, C. E., Characterization of Volatile Organic Compounds in Human Leukocyte Antigen Heterologous Expression Systems: a Cell's “Chemical Odor Fingerprint”. Chembiochem 2012, 13 (7), 1053-1059.
5. 5.Ray, A.; Bristow, T.; Whitmore, C.; Mosely, J., On-line reaction monitoring by mass spectrometry, modern approaches for the analysis of chemical reactions. Mass Spectrom Rev 2018, 37 (4), 565-579.
6. Luchner, M.; Gutmann, R.; Bayer, K.; Dunkl, J.; Hansel, A.; Herbig, J.; Singer, W.; Strobl, F.; Winkler, K.; Striedner, G., Implementation of proton transfer reaction-mass spectrometry (PTR-MS) for advanced bioprocess monitoring. J Biotechnology and Bioengineering 2012, 109 (12), 3059-3069. Discloses PTR-MS used to correlate VOCs with culture growth.

Despite the above, there remains a void in regard to the chemical species and quantity of VOCs produced by cells in laboratory cell expansion. In addition, the practical problems of monitoring VOCs in-process, such as maintaining sterility if samples are taken, have not been addressed.

### INVENTION SUMMARY

The inventors have recognised the above problems and have also realised that it is possible to correlate VOC profiles from bioreactors with cell density over a significant time period of cell expansion, using non-invasive methods. Their findings show that, for example, for both CHO and T cells, which are important cell expression models for use in bioprocess engineering and cellular immunotherapy workflows, respectively, it is possible to estimate cell numbers using VOC profiles, particularly where VOCs are monitored over time, and utilize the estimated cell numbers to control process parameters. The estimated cell numbers, over time, also provide an indication of cell viability, health, and/or nutrient utilization.

Herein, the term Volatile Organic Compounds (VOCs) includes organic compounds which are dissolved or suspended in a solid, liquid or gas (including vapour or droplets suspended in a gas), as well as organic compounds which and classed as semi-volatile (SVOCs).

The disclosure herein, in summary, provides details of how cell emissions of VOCs were measured from Chinese Hamster Ovary (CHO) cell and T cell bioreactor wastes with the goal of non-invasively metabolically profiling the expansion process. Measurements were made, for example, directly from the gas exhaust lines using sorptive elements, in this case polydimethylsiloxane (PDMS)-coated magnetic stir bars, which underwent subsequent gas chromatography-mass spectrometry (GC-MS) analysis. Baseline VOC profiles of the cell cultures were observed from bioreactors filled with only liquid media (i.e. without cells), and unique VOC profiles correlated to cell expansion over the course of 8 days. Partial least squares (PLS) regression models were built to predict cell culture density based on VOC profiles of CHO and T cells (R2=0.671 and R2=0.769, respectively, based on a validation data set). T cell runs resulted in 47 compounds relevant to cell expansion while CHO cell runs resulted in 45 compounds; the 20 most relevant compounds of each cell type were putatively identified. On the final experimental days, sorbent-covered stir bars were placed directly into cell-inoculated media and into media controls. Liquid-based measurements from spent media containing cells could be distinguished from media-only controls, indicating soluble VOCs excreted by the cells during expansion. A PLS discriminate analysis (PLS-DA) was performed, and 96 compounds differed between T cell-inoculated media and media controls with 72 compounds for CHO cells. The 20 most relevant compounds of each cell line were putatively identified. This work demonstrates that VOC-based detectors can be incorporated in bioreactor gas and liquid waste volumes to non-invasively monitor cellular health and to optimize cell expansion conditions in real time with appropriate control systems. For example, by monitoring cell expansion over time based on the intensity of VOC, an indication of cell viability, health, and/or nutrient utilization can be provided.

The invention, according to one aspect, provides a method for monitoring cell density during cell expansion resulting from a cell culture process in a bioreactor comprising the steps of:
a) cultivating cells in a bioreactor culture chamber according to a cell culture process having cell culture parameters;
b) during said process, introducing cell culture fluid inputs and generating waste materials;
c) determining an intensity of volatile organic compounds (VOCs) and their chemical species in the waste materials; and
d) estimating the density or population of cells in the bioreactor based on said determination.

The method may further include a step of:
e) control at least one process parameter related to the cell culture process based on the estimating step.

The method many further also include a step of:
f) providing an indication of cell viability, health, and/or nutrient utilization based upon the estimated density or population of cells over time.

In an embodiment said waste materials include bioreactor headspace gases, and/or filtered liquid waste, and said VOCs include gas phase and/or dissolved or suspended VOCs respectively.

In an embodiment, the waste materials are isolated or removed from the bioreactor chamber prior to said determining.

In an embodiment, said isolation is achieved by an isolation filter allowing only the passage of gases out of the chamber and inhibiting the passage of contaminants into the chamber.

In an embodiment, during or after said process, the VOCs are collected from said waste materials prior to said determining.

In an embodiment, said collecting includes exposing the waste materials to a collective element, such as chemical adsorption or absorption element, and said determining step includes subjecting the collected chemicals to a detector element, for example mass spectrometry (MS) or proton transfer reaction MS, to provide said intensity and profile of VOCs.

In an embodiment, said collecting and said determining are conducted continually, periodically or intermittently.

In an embodiment, said estimating includes assessing the change, and/or rate of change of the VOC concentration/profile.

In an embodiment, said cells are CHO or T cells and the estimation of cell density includes the measurement of the concentration of one or more of alkanes, alkenes, alkynes, carbonyls, esters, alcohols, arenes, acids, amides, amines, carbohydrates, steroids, proteins, nucleic acids and oximes.

In an embodiment, said measurement includes the measurement of the increase in concentration of VOCs, for example, docosane and/or other alkanes.

In an embodiment, a) where said cells are CHO cells, then the measurement includes the measurement of the decrease in concentration of VOCs or b) where said cells are T cells, then the measurement includes the measurement of the decrease in concentration of VOCs, for example, benzaldehyde and/or other aldehydes.

In an embodiment, the ratio of VOCs, for example the ratio of measured alkanes, alkenes, alkynes, carbonyls, esters, alcohols, arenes, acids, amides, amines, carbohydrates, steroids, proteins, nucleic acids and oximes, is used to determine cell density/concentration.

In an embodiment, e) control of at least one process parameter related to the cell culture process includes altering or enhancing cell culture parameters and/or cell culture fluid inputs.

In an embodiment, e) control of at least one process parameter related to the cell culture process includes adjusting chemical and biophysical parameters to further increase expansion, inform harvesting decisions, and control the chemical environment through culture media changes.

The invention, according to a further aspect, provides a cell culture system arranged for monitoring cell density during cell expansion resulting from a cell culture process; the system comprising:
a) a bioreactor including a culture chamber suitable for cultivating cells;
b) a controller for conducting a cell culture process according to cell culture parameters;
c) at least one cell culture fluid input and at least one waste materials output;
d) one or more VOC sensors or collectors present in or at the waste output; and
e) means for determining the intensities of VOCs sensed or collected and their chemical species.

The controller may be further configured to estimate the density or population of cells in the bioreactor based on the determined the intensities of VOCs sensed or collected and the specific combination of the specific chemical species.

The controller may be further configured to provide an indication of cell viability, health, and/or nutrient utilization based upon the estimated density or population of cells over time.

The system may further comprise:
e) means to control at least one process parameter related to the cell culture process based on the estimated the density or population of cells.

In an embodiment, said at least one waste materials volume includes: a bioreactor headspace for head space waste gases, a waste gas outlet, an area in the chamber where waste fluids collect, a fluid waste collection line or vessel, a fluid circulation line, and/or a solid waste collection line or vessel.

In an embodiment, said one or more VOC collectors include a collection element such as a sorptive element at least partially within the waste materials volume.

In an embodiment, the system further includes an isolation filter allowing only the passage of gases out of the chamber and inhibiting the passage of contaminants into the chamber, and wherein said waste material volume is downstream of said filter thereby isolating the volume from the chamber.

In an embodiment, means for determining the intensity of VOCs collected and their chemical species is a chemical detector, for example mass spectrometry (MS) or proton transfer reaction MS.

In an embodiment, means to control at least one process parameter related to the cell culture process based on the estimation is said controller, the controller being adapted to alter the cell culture parameters in response to the determination of the intensity of VOCs collected and their chemical species and an estimated density or population of cells in the bioreactor based on the determined intensity of VOCs.

In an embodiment, the controller is adapted to adjust chemical and biophysical parameters to further increase expansion, inform harvesting decisions, and control the chemical environment through culture media changes.

The invention extends to any combination of features disclosed herein, whether or not such a combination is mentioned explicitly herein. Further, where two or more features are mentioned in combination, it is intended that such features may be claimed separately without extending the scope of the invention.

### BRIEF DESCRIPTION OF THE DRAWINGS

The invention can be put into effect in numerous ways, illustrative embodiments of which are described below with reference to the drawings, wherein:

FIG. 1a shows schematically a typical bioreactor system;

FIGS. 1b,c,d and e show the bioreactor of FIG. 1a in use at different times;

FIG. 2 shows graphical principal components analysis (PCA) results for VOC emissions—in more detail, PCAs of headspace volatile compound emissions from four bioreactors (two CHO, two T cell cultures). Cell culture samples are sized by day of expansion (smallest: Day 1, largest: Day 8). A) Comparison of bioreactor bag & gas controls, media controls and cell culture samples, which separated along PC 1. B) Cell culture samples during the eight days of expansion exhibited a VOC profile change along PC 1;

FIG. 3 shows graphically the correlation between predicted and experimentally obtained cell count results, in more detail—PLS regression models built from VOC profiles of A) CHO cells and B) T cells. Samples were randomly split into 66% calibration and 33% validation (test) sets. Cell counts are reported per mL of media;

FIG. 4 shows graphically the change in content (Y axis) over days (X axis) of certain volatile groups obtained from a bioreactor—in more detail, the graphs show how the 20 VOCs most relevant to cell culture expansion changed over 8 days. Compounds were split into 4 clusters via hierarchical clustering. VOCs in each cluster are found in Table 1 and are presented as normalized to the maximum intensity within a compound (Norm. Inten.). A) CHO cells B) T cells. Each point is the average of n=8 replicates (4 technical replicates×2 biological replicates).

FIG. 5 shows graphically a decrease in content (Y axis) over days (X axis) of certain volatile groups obtained from a bioreactor, in more detail—VOCs that decreased during cell expansion (from Cluster 4, FIG. 4), including gas & bag (G&B) controls and media controls. A) CHO cells B) T cells. Each point is an average of n=8 replicates (4 technical replicates×2 biological replicates);

FIG. 6 shows graphical principal components analysis results for dissolved VOC in liquid media from media control and form inoculated media;

FIG. 7 shows the viable cell density measured according to conventional techniques, measured during the experimentation illustrated in the Figures above; and

FIG. 8 shows the cell culture metabolites measured over the same cell culture period as measured in the graph of FIG. 7.

### DETAILED DESCRIPTION OF THE INVENTION

The invention, together with its objects and the advantages thereof, may be understood better by reference to the following description taken in conjunction with the accompanying drawings, in which, like reference numerals identify like elements in the Figures.

### Cell Culture Methodology

Primary T cells were isolated from buffy coats (sourced from Canadian Blood Services) from 2 donors using a Ficoll density gradient and cultured in T flasks for 6 days prior to inoculation in a Xuri Cell Expansion System (CES, GE Healthcare) at ˜7×10⁵ cells/mL in 1 L of T cell culture medium. T cell culture medium was Xuri Expansion Medium (GE Healthcare) with 1% penicillin-streptomycin (Hyclone), 5% human AB serum (GemCell), and 350 IU/mL Xuri IL-2. CHO-M cells (courtesy of GE Healthcare, Uppsala, Sweden) were cultured in T flasks in ActiPro (Hyclone) medium with 1% penicillin-streptomycin and 2 mM L-glutamine (Hyclone). CHO cells were inoculated in a Xuri CES at ˜2×10⁵ cells/mL in 1 L.

Four 2 L Xuri Cellbags (working volume of 1 L each) with dissolved oxygen (DO) and pH sensors were connected to Xuri CESs. The 2 L Cellbag was inflated with compressed air and 5% CO² and then left overnight with 200 mL culture medium to equilibrate the DO/pH sensors. Temperature was set to 37° C. and the platform set to rock at 10 rocks per minute (rpm) at a 6° angle. For two minutes in each hour, the platform rocked at 2 rpm at a 2° angle. Perfusion was initiated using a step-wise protocol based on a combination of lactate measurements as well as cell density. Below 2×10⁶ cells/mL, no perfusion was initiated. Above 2×10⁶ cells/mL, medium was perfused at 0.5 L/day at VCD between 2×10⁶-10×10⁶ cells/mL, at 0.75 L/day for VCD between 10×10⁶-15×10⁶ cells/mL, and at 1 L/day for VCD greater than 15×10⁶ cells/mL. A 1 L/day perfusion was initiated regardless of the VCD in the event of a lactate concentration exceeding 20 mM.

### Bioreactor VOC Exhaust Measurements

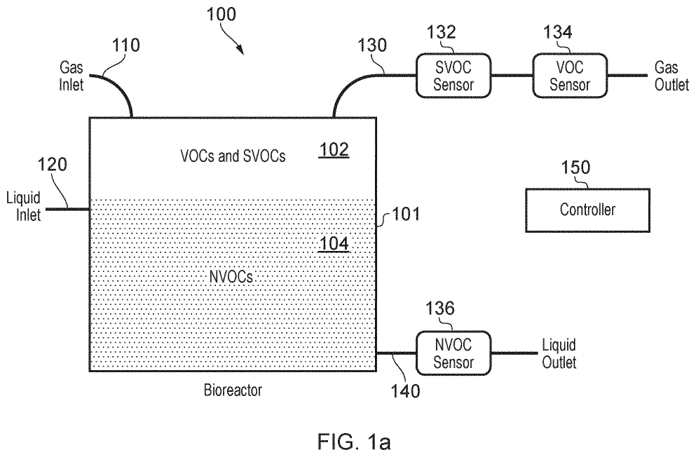

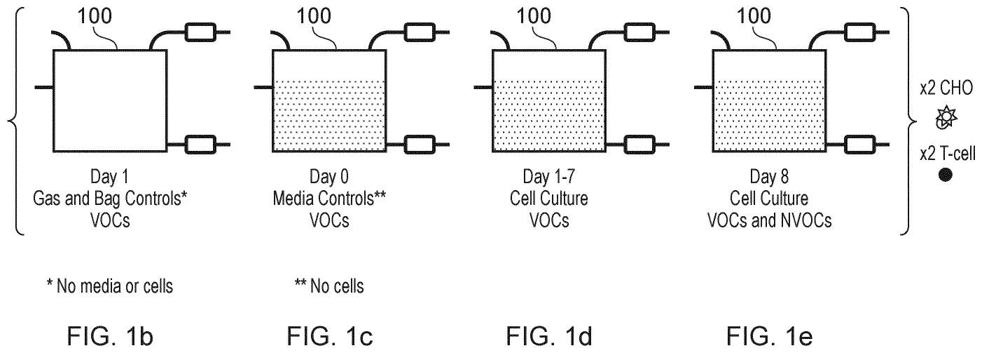

FIG. 1a shows schematically an example of a cell culture bioreactor for use with the invention. Therein, a bioreactor 100 is shown as a rectangular rigid generally closed vessel 101, although flexible bag type bioreactors commercially available under the brand name of Xuri Cellbags as mentioned above and vessels with semipermeable membrane walls could also be employed. In most cases gas and liquid inlets 110/120 are used to introduce oxygen, cells and cell nutrients, and in some systems recirculate cells which have been separated from waste materials in a filter or the like, removed from the bioreactor using a liquid waste line 140. Waste gas can be removed via a gas outlet 130 to make way for new gas via the inlet 110. In the experiments described in more detail below, cell culture VOC emissions from the gas exhaust (waste) line of bioreactors were measured using a head-space sorptive element (HSSE) technique. In another embodiment, known VOC sensors 132 and 134 could be used with equal utility, and would then provide real-time monitoring of VOCs, and where the range of sensing is limited, SVOC monitoring also. Further VOCs can be measured in the waste liquid outlet 140 by an alternative sensor 136. Such sensing could include non-volatile OCs also. The bioreactor in use will contain a liquid phase cell culture 104 and a gas headspace 102. The bioreactor will be under the control of a controller 150, which could be local or remote and may be shared.

Bioreactor air exhaust was directed via PTFE tubing through the lid of a capped borosilicate jar. Each bioreactor employed was connected with a single jar and the same jar was used throughout the course of the entire experiment. Each jar contained four sterile and pre-conditioned HSSE stir bars (“Twisters®”, Part 011222-001-00, Gerstel US, Linthicum Heights, Md.), held in place to the side of the jar by magnets, providing four technical replicates per sample. The commercially available HSSE bars were 10 mm in length and contained a 0.5 mm thickness of polydimethylsulfide (PDMS) sorbent. Twisters® were left to extract cell culture VOCs in 24 h increments. After this period, the lids were removed from the jars, the four Twisters® were collected and replaced with four fresh HSSE bars, and the lid was screwed back onto the jar.

### Liquid-Phase In Situ VOC Measurements

A final time point measurement to examine VOCs dissolved in the liquid media was made using Twisters® in a stir bar sorptive extraction (SBSE) immersion technique. This was not performed until the end of the experiment to reduce the risk of cell culture contamination. During the final 24 h of the experiment, four sterilized Twisters® (soaked in 70% ethanol for 10 min) were dropped directly into each cell culture via a port on the CellBag bioreactor. Once extraction was complete (24 h), the bioreactor bags were sliced open and the Twisters® were collected. The experiment ended at this point and cells were destroyed. For media only controls, additional Twisters® were placed directly into 20 mL of cell-free media of each type for 24 h and incubated at the same temperature as the cultures.

### Time Course Explanation

FIGS. 1,b,c,d and e show schematically the bioreactor system employed for the culturing mentioned above, illustrated in use at different times during the cell culture process—about 8 days in this instance. The day prior to media equilibration (FIG. 1b Day-1), four empty Xuri CellBags were attached to the Xuri units with air flow (compressed air+5% CO2) on and “bag and gas controls” were collected to measure background VOCs. The day of media addition (FIG. 1c Day 0), two bioreactors had 200 mL T cell media added and two reactors had 200 mL CHO media added; “media controls” were collected (no media perfusion during this day). On the day of cell seeding (FIG. 1d Day 1), the bioreactors were inoculated with their respective cell lines. HSSE VOC measurements were conducted over 8 days of cell expansion. On day 8 (FIG. 1e), the liquid SBSE measurements and HSSE measurements were concurrently collected. Every 3-4 d, four unused Twisters® were pulled aside for “sorbent controls” which acted as shipping and handling controls to ensure VOCs of unknown origin did not compromise the experiment.

Twice a day, an aliquot (5-10 mL) from the bioreactors was collected for measurements of culture attributes/metabolites: viable cell density (VCD), % viability, glutamine, glutamate, glucose, lactate, ammonium, sodium, potassium, calcium, pH and pO2. VCD and viability were measured on a Nucleocounter NC-200 (Chemometec, Allerod, Denmark). Metabolite measurements were conducted on a BioProfile FLEX 2 Analyzer (Nova Biomedical, Waltham, Mass.).

Twister®-GC-MS Analysis

There were 2 biological replicates for T cells and 2 technical replicates for CHO cells, with 4 technical replicates of each per time point. All Twisters® were pre-conditioned prior to use, according to manufacturer specifications.

As soon as Twisters® were extracted from the cell culture reactors, they were placed into 2 mL borosilicate vials and an aliquot of the first internal standard (1 μL of a 1 ppm naphthalene-D8 in ethanol solution) was pipetted into each vial. Twisters® were kept frozen until analysis. Just prior to analysis, they were transferred into thermal desorption tubes alongside an aliquot of the second internal standard (1 μL of a 0.1 mL/L decane-D22 in ethanol).

Individual Twisters® were thermally desorbed using a thermal desorption unit (TDU, Gerstel US) and cooled injection system (CIS, Gerstel US). The TDU was initially set to 30° C. for 0.5 min and heated at 60° C./min until reaching 300° C. and held for 3 min. A flow of helium led desorbed analytes into the CIS, which was held at −80° C. After desorption, the CIS heated at 12° C./s to 300° C. and was held for 3 min. This process splitlessly injected analytes onto the head of the GC column.

Chromatography occurred on an Agilent 7890A GC (Agilent Technologies Inc., Santa Clara, Calif.) equipped with a DB-5 ms column (30 m×250 μm×0.25 μm, Agilent Technologies Inc.). The column was initially at 35° C. for 3 min, then heated at 2° C./min to 200° C., then heated at 30° C./min to 300° C. and held for 5 min. Total runtime was 93.8 min. The GC was operated in constant flow mode (1.5 mL/min of helium). Analytes eluted into a 5975C single quadrupole mass spectrometer (MS, Agilent Technologies Inc.). The MS scanned from 33 to 300 m/z. Its source and quad were set to 230° C. and 150° C., respectively.

A bake out of the TDU-CIS-GC-MS system was conducted every ˜20 injections. After every 30-40 GC-MS injections, a standard mixture of C8-C24 alkanes was analysed to serve as an external 20 control of the instrument and also to calculate Kovats retention indices of compounds.

### GC-MS Data Processing

GC-MS data files were deconvoluted and aligned using the recursive feature extraction on Profinder (Version B.08.00, Agilent Technologies Inc.). Peak areas were normalized to the first internal standard. Features with siloxane base peaks (73, 147, 207, 221 and 281 m/z) were removed. Statistical analyses were performed using GeneSpring (Version B.14.9, Agilent Technologies Inc.) and PLS Toolbox (Version 8.6, Eigenvector Research Inc., Manson, Wash.). A p-value of p<0.05 was used throughout for significance. Putative peak identification was possible through spectral matching with the NIST 14 mass spec database along with comparison of calculated Kovats Retention Index comparisons to reported literature values.

To model changes in VOC profiles related to cell growth, HSSE data from both CHO cell reactors were pooled together and VOC data from both T cell reactors were pooled together, and data were autoscaled. Within each of these two groups, the data were randomly separated: 67% for a calibration training set and 33% for a validation set. Partial least squares regression (PLS) was applied to correlate live cell densities (the Y space) to the VOC profiles (the X space) using PLS_Toolbox software (Eigenvector Research Inc., Manson, Wash.). Cross-validation was performed using the venetian blinds technique, where the calibration data were split into 10 random splits and one sample per split was used to cross-validate the model. To cluster compounds of similar changes in intensity, agglomerative hierarchical clustering was applied using the shortest distance algorithm in MATLAB R2017a software (MathWorks, Natick, Mass.).

SBSE data were divided into the two cell types and their respective controls. A PLS-discriminate analysis (PLS-DA) was performed on each cell type to categorically distinguish media controls from cell samples.

### Results & Discussion

### Cell Expansion

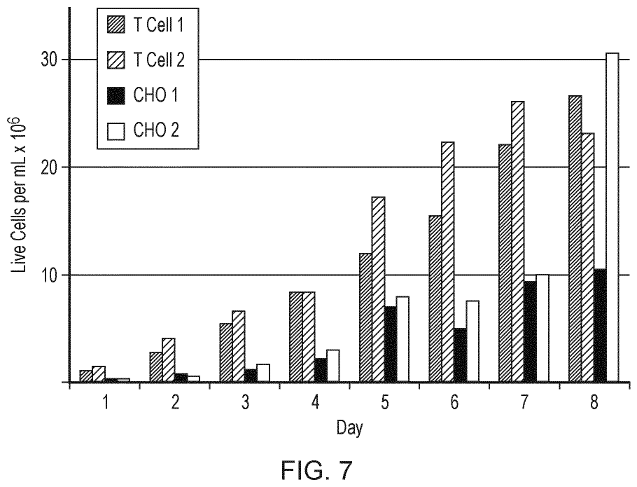

At the time of media inoculation, the concentrations of CHO cells were 2.2×10⁵ and 2.6×10⁵ cells/mL per reactor respectively, and T cells were 7.0×10⁵ and 8.0×10⁵ cells/mL (FIG. 7). By the end of the experiment, the majority of the bioreactors increased cell density by 16-30 times indicating exponential growth over the culture duration in the Xuri CES. On the final day of the experiment, one of the CHO reactors (CHO 2) experienced an unrelated technical issue and lost much of its media, resulting in a sudden spike in cell density for the CHO 2 reactor on day 8. These samples were removed from the subsequent PLS regression analysis (see below).

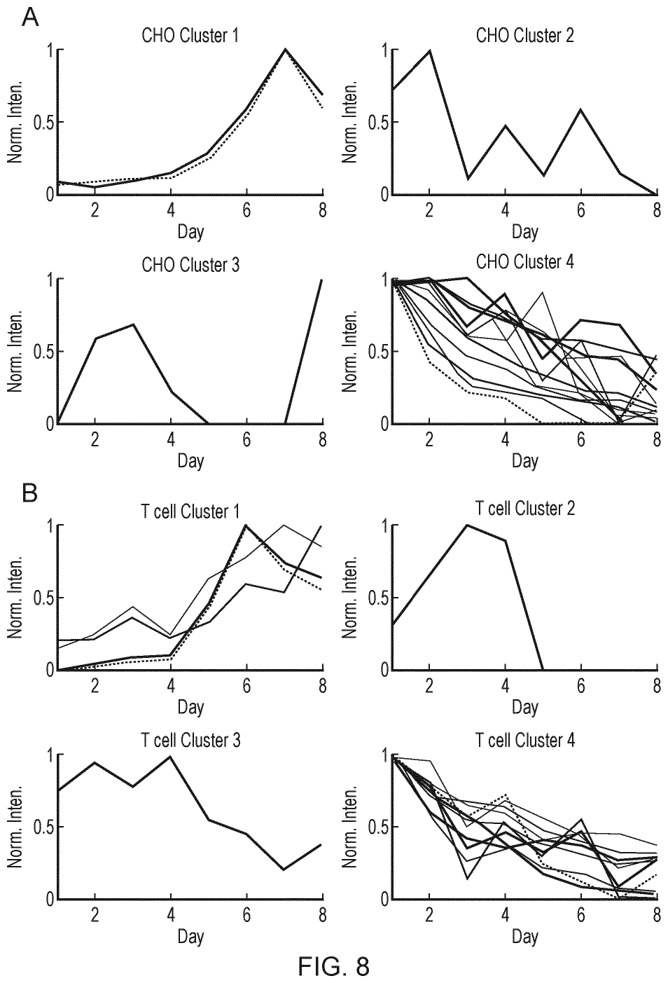

Measured metabolites are also provided in FIG. 8 for the duration of culture in the Xuri CES. Monovalent and divalent cations such as K+, Ca2+, and Na+ had fairly stable levels throughout the experiment. As expected, during the initial days of culture in the Xuri CES, pO2, glutamine and glucose concentrations dropped as these metabolites were consumed and lactate and ammonia rose as these byproducts were accumulated. Similarly, a concomitant decrease in pH was observed over the course of the early days of culture corresponding to an increase in lactate. After perfusion was initiated, nearly all metabolites attained steady state levels.

### VOC Profiles of Downstream Bioreactor Emissions

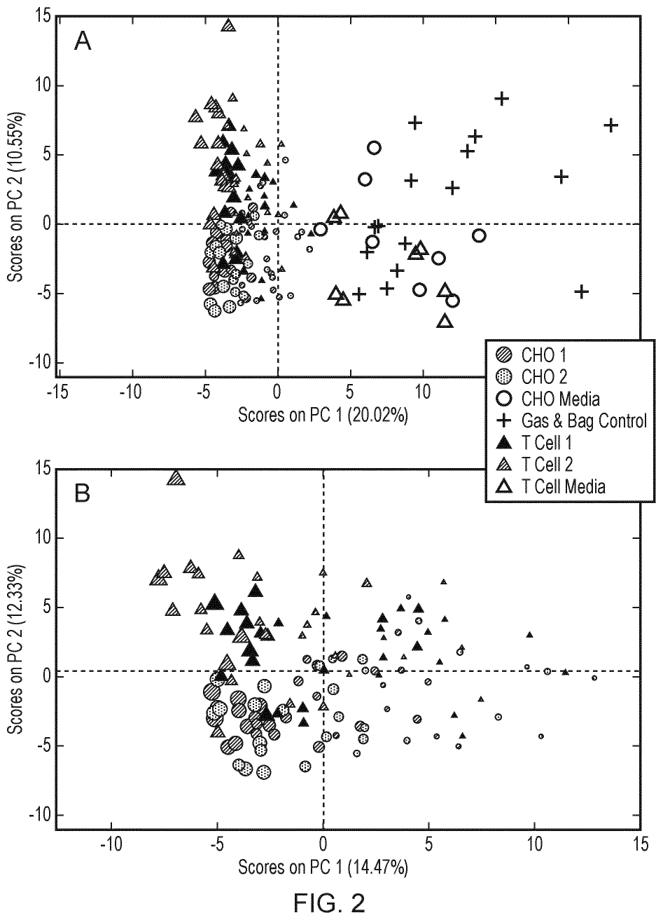

Principal components analysis (PCA) was applied to all HSSE samples (FIG. 2 top graph). VOC profiles of the two control types (media, gas and bag) differed from bioreactors containing cells. Cell samples separated from controls along PC 1, which explained 20.02% of the variance. PCA is an unsupervised method that does not take into account meta-information about the sample (such as sample treatment or type) in its analysis. Instead, PCA only plots the variation between the GC-MS samples. Having control samples separate from cell samples along the first principal component suggests that the bioreactors with CHO and T cells exhaust cellular VOCs in levels that make them distinguishable from bioreactors filled with only media.

In addition to separating from controls, there was a trend for cell types to separate (FIG. 2 bottom graph). T cell samples had a tendency to separate from CHO cell samples along PC 2, which explained 12.33% of the variance, indicating unique VOC profiles among the cell types. More interesting was the gradual shift of samples that occurred along the PC 1, which explained 14.47% of the variance. PC 1 showed strong correlation to experimental day. With the bioreactors controlling all of the conditions of the reactor (gas flow, media perfusion, temperature, etc.), the shift along PC 1 is strongly suspected to correlate to viable cell density, which increased with experimental day (FIG. 7).

Prior to any statistical analysis, including PCA, samples were normalized to the internal standard. This practice would account for any potential signal drift caused by the GC-MS instrument. Further, visualization of the internal standards results do not suggest an instrument drift occurred (data not shown), confirming that changes in the VOC profile must have related to changes in the bioreactor.

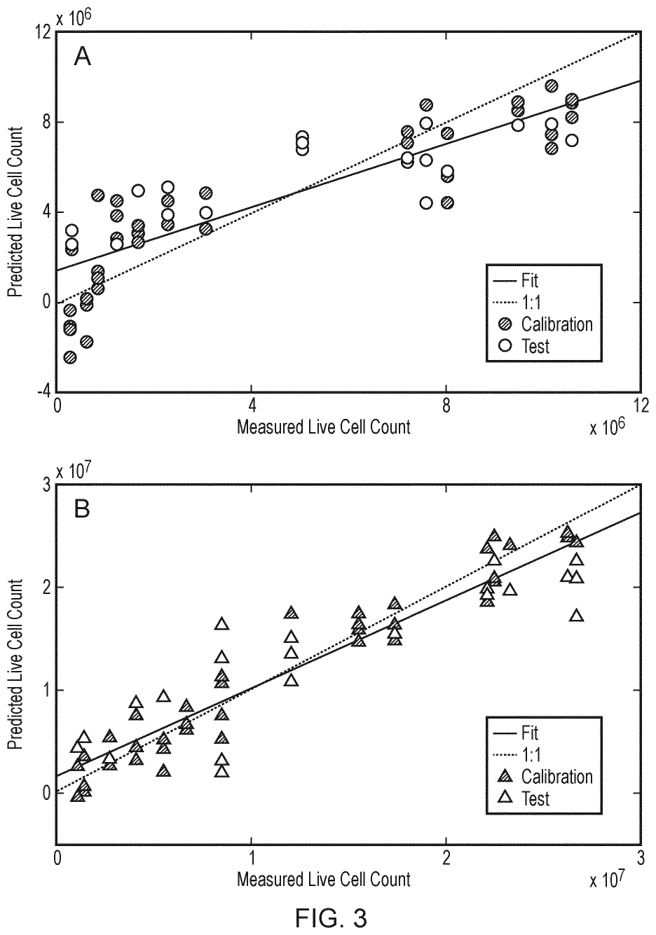

To correlate cell growth to VOC profiles, two PLS regression models were built, one for CHO cells and one for T cells. Within each cell type, 67% of data were used to train and calibrate the PLS model, which was then applied to the remaining 33% as a blinded validation set. Models showed a correlation between the live cell density and the VOC profiles collected using the HSSE-GC-MS extraction technique (FIG. 3). Based on R2 values, the T cell model had a slightly better linear fit, relative to CHO cells (Table 1); although both cell models performed very well with high R2 values. As a measure of accuracy, T cells had slightly higher root-mean-square error (RMSE), even when normalized to the range of cell counts (maximum cell count minus minimum). In the validated sets, T cells had more than twice the normalized RMSE than CHO cells, although in general all of these MRSE values are fairly low.

TABLE 1
Linear correlations (R2), root-mean-square errors
(RMSE) and normalized RMSE (NRMSE, normalized
to cell count range) from the two PLS models relating
VOC profiles to live cell density (FIG. 3).

| | CHO cells | T cells |
|---|---|---|
| R² Cross-validation set | 0.724 | 0.842 |
| RMSE Cross-validation set | 2.04 × 10⁶ | 3.47 × 10⁶ |
| NRMSE Cross-validation set | 1.98 × 10⁻¹ | 3.37 × 10⁻¹ |
| R² Validation set | 0.671 | 0.769 |
| RMSE Validation set | 2.12 × 10⁶ | 4.53 × 10⁶ |
| NRMSE Validation Set | 2.06 × 10⁻¹ | 4.40 × 10⁻¹ |

In a PLS analysis, variable importance in projection (VIP) scores are generated for each variable (in this case, a chemical VOC of interest). Variables with a VIP score greater than 1 are typically considered relevant to the regression. T cells had 47 compounds with a VIP>1 , and CHO cells had 45 compounds; 26 compounds overlapped between the two cell lines.

Putative identifications were made on the 20 compounds with the highest VIP score for the T cell model and the 20 compounds with the highest VIP score for the CHO model (Table 2). 27.0% of these compounds were classified as a type of alkane, while 15.4% were esters, 7.7% alcohols, 7.7% oximes, and 23.0% others with 19.2% unknown.

By using HSSE-GC-MS, we believe we are the first group to report the identities of VOCs emitted by CHO and T cells in a bioreactor during cell expansion. Without other studies to offer comparison, we compare these results to other cell culture experiments and find that the types of VOCs identified in this work are in general agreement. 2-ethyl-1-hexanol was found relevant to viral infections of human laryngeal cancer cells.16 Benzaldehyde has been observed in emissions of human fibroblasts (hFB). 17 Esters have been observed in cultures of human B-lymphoblastoid cells. 18 Alkanes and alcohols have been observed in epithelial cell cultures. 15 While known background compounds were not included in statistical analyses, such as siloxanes from the PDMS sorbent and GC column bleed, phthalates might be artefacts from the plastics within the bioreactor system.

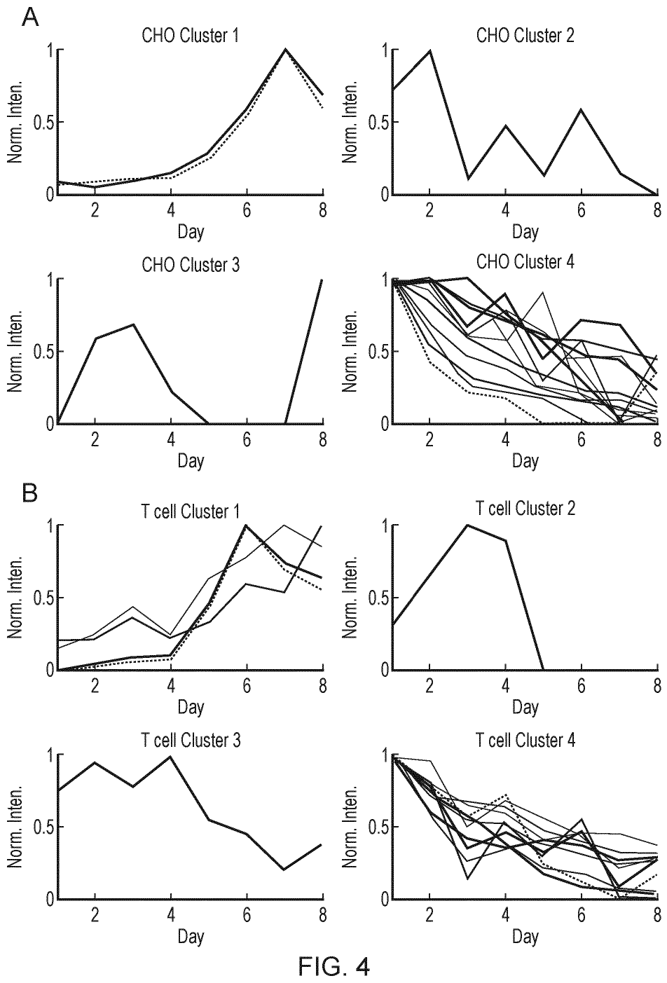

TABLE 2
Based on downstream bioreactor VOC emissions. Putative identifications of the 20
compounds with the highest VIP scores for the T cell regression model and the 20
compounds with the highest VIP scores for the CHO cell regression model (FIG. 3),
combined into one table. KI: Kovats index, calculated (Calc) and as reported in
the literature (Lit); MS Score: Score of acquired mass spectrum compared to the
NIST mass spectral database; Cluster: group applicable to the clusters in FIG. 4.

| | | | | | VIP Score | | | |
|---|---|---|---|---|---|---|---|---|
| | KI | KI | | MS | (if > 1) | | Cluster | |
| Compound | (Calc) | (Lit) | CAS # | Score | T cells | CHO | T cell | CHO |
| undecane | 1100 | 1100 | 1120-21-4 | 93.71 | 2.57 | 2.69 | 4 | 4 |
| unknown 1 (alkane) | 1170 | | | | 2.49 | 2.77 | 4 | 4 |
| 2-(2-hydroxyethoxy)ethyl | 1124 | | 1000351-92-4 | 83.52 | 2.46 | 2.70 | 4 | 4 |
| acetate | | | | | | | | |
| unknown 2 (alkane) | 1097 | | | | 2.37 | 2.65 | 4 | 4 |
| 2-ethylhexanal | 952 | 955 | 123-05-7 | 82.42 | 2.20 | 2.20 | 4 | 4 |
| docosane | 2206 | 2200 | 629-78-7 | 89.69 | 2.17 | 2.59 | 1 | 1 |
| unknown 3 (alkane) | 2220 | | | | 2.13 | 2.47 | 1 | 1 |
| unknown 4 | 1169 | | | | 2.12 | 1.57 | 4 | 4 |
| 2-ethyl-1-hexanol | 1033 | 1029 | 104-76-7 | 96.06 | 1.98 | | 3 | |
| diisobutyl phthalate | 1863 | 1868 | 84-69-5 | 76.01 | 1.92 | 1.29 | 2 | |
| unknown 5 | 969 | | | | 1.84 | | 4 | |
| unknown 6 | 1170 | | | | 1.80 | | 4 | |
| unknown 7 (phthalic | 2202 | | | | 1.74 | | 1 | |
| acid, alkane ester) | | | | | | | | |
| 2-methyldecane | 1062 | 1065 | 6975-98-0 | 84.10 | 1.66 | 1.85 | 4 | 4 |
| unknown 8 | 1345 | | | | 1.65 | 1.21 | 1 | |
| decane | 1001 | 1000 | 124-18-5 | 72.22 | 1.64 | 1.98 | 4 | 4 |
| benzaldehyde | 955 | 958 | 100-52-7 | 60.04 | 1.57 | 1.47 | 4 | 2 |
| unknown 9 (haloalkane) | 950 | | | | 1.55 | 1.65 | 4 | 4 |
| 1-methyl-4-propyl-2- | 1050 | 993 | 33063-77-3 | 55.06 | 1.54 | 1.99 | 4 | 4 |
| pyrazoline | | (est) | | | | | | |
| methoxyphenyloxime | 943 | | 1000222-86-6 | 65.25 | 1.53 | 1.64 | 4 | 4 |
| methoxyphenyloxime (2) | 939 | | 1000222-86-6 | 68.71 | 1.08 | 2.53 | | 4 |
| 1-dodecanol | 1475 | 1469 | 112-53-8 | 79.75 | 1.04 | 2.01 | | 4 |
| 1,2-dibutoxyethane | 1190 | 1144 | 112-48-1 | 69.59 | | 1.72 | | 4 |
| unknown 10 | 1251 | | | | | 1.80 | | 3 |
| unknown 11 (ketone) | 1154 | | | | | 1.81 | | 4 |
| 1(3H)-isobenzofuranone | 1335 | 1272 | 87-41-2 | 87.64 | | 1.56 | | 4 |
| | | (est) | | | | | | |

Some compounds increased in intensity with cell expansion while others decreased. To group compounds by patterns of change, hierarchical clustering was applied to the top 20 CHO and 20 T cell compounds from Table 2. Each dendrogram was divided in such a way to yield four clusters of VOCs. Each cluster was plotted to demonstrate the compounds' intensities over the course of the 8 d of cell expansion (FIG. 4). Both CHO and T cells exhibited compounds that increased over the course of cell expansion (Cluster 1 compounds). Two compounds increased over time in both cell lines: docosane and an unidentified alkane. Both cell types had a compound that increased until Day 3-4, and then suddenly disappeared (CHO: Cluster 3, unknown 10; T cell: Cluster 2, benzaldehyde). The compounds that increased over time are likely direct emissions from the cell cultures. These compounds could be directly monitored and exploited in a VOC-based PAT. By measuring downstream VOC emissions, there is no risk to contaminate the cell cultures, as is currently the case with withdrawing 5-10 mL from the reactor to manually measure cell count. VOC-based PAT could provide substantial cost savings with its non-invasive ability to assess cell culture health.

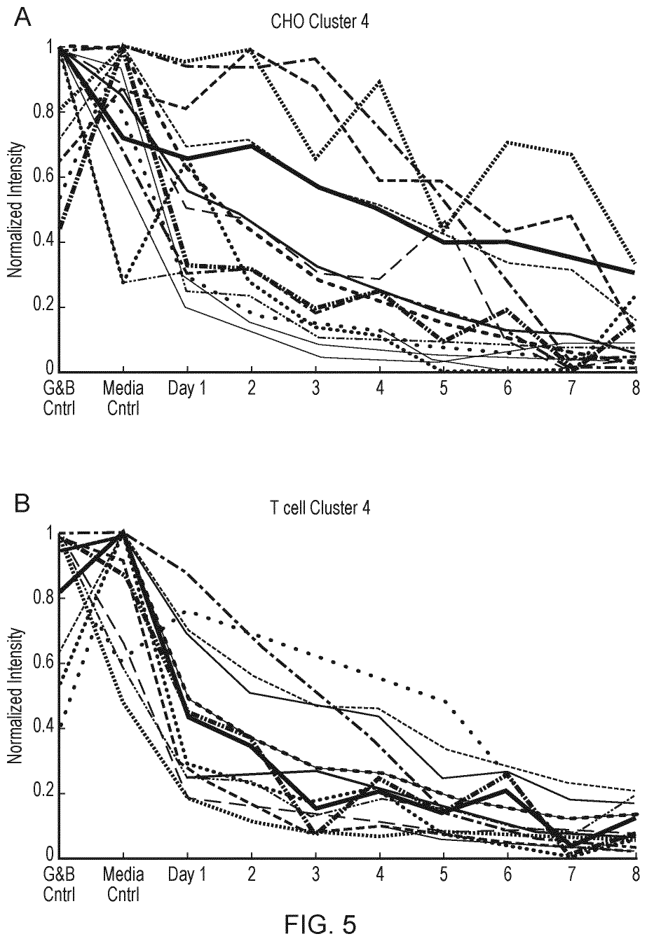

The majority of these most relevant VOCs decreased during cell expansion (Cluster 4 compounds, FIG. 4). FIG. 5 includes the gas and bag controls and media controls with these decreasing compounds. All compounds were present in bioreactor controls prior to introduction of cells. Thus, it is possible that the cultures are metabolizing these compounds during expansion. Although media perfusion is occurring, this rate might not be fast enough to replenish these compounds as quickly as the cells are consuming them. This provides another opportunity for VOC exploitation: in addition to monitoring VOCs emitted by the cell cultures, it is possible to monitor the nutrients found in the media and adjust perfusion rates to provide sufficient growth material for optimal cell growth.

### Liquid-Phase VOC Profiles of Cell Cultures

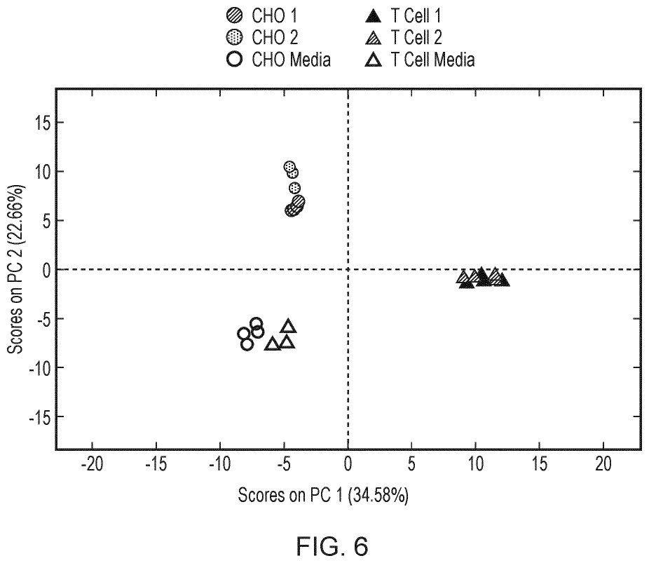

SBSE measurements made directly in bioreactor bags isolated more cellular VOCs from media controls than HSSE measurements of bioreactor gas exhaust. A PCA of these liquid-phase extractions (FIG. 6) showed clear differences between the two cell types and media controls, which separated between PC 1 and PC 2, explaining a total of 57.24% of the variance.

Two PLS-DA analyses were performed that distinguished liquid media controls from respective cell lines. Similar to PLS regression, each variable (in this case, chemical VOC compound) was assigned a VIP score. CHO cells had 72 compounds with a VIP score >1 and T cells had 96 compounds, with 43 overlapping between cell lines. T cells had 16 compounds with VIP scores >1 in both downstream VOC emission measurements (HSSE) and cell-inoculated liquid measurements (SBSE); there were 9 such compounds for CHO cells.

The 20 compounds with the highest VIP scores for each cell types were putatively identified (Table 3). Not all these compounds were present in liquid media controls. Compared to HSSE, SBSE extracted more compounds of higher molecular weights. Many contain aromatic rings (toluenes, phenols, benzoic acids, benaldehydes, acetophenones, etc.). One compound, unknown 10, appears in both Table 2 and Table 3, having importance only in CHO cells in both HSSE and SBSE measurements.

Some compounds appear related to the mevalonate pathway. Important to cell membrane function and steroid synthesis, cholesterol was putatively identified in both CHO and T cell bioreactors. A derivative of citronellol was found in CHO cells, which may be a hydrogenated product of geraniol, a compound involved in cholesterol synthesis pathways. 19 P-benzoquinone could be attributed to exposure to benzene derivatives or as a breakdown product of ubiquinone. Naphthols such as 1-amino-2-naphthalenol may derive from biomarkers related to exposure to polycyclic aromatic hydrocarbons, such as plasticizers. 20 Heretocyclic compounds such as quinazolines, quinolinones and pyrazoles may have resulted from other steroids.

TABLE 3
Based on measurements made directly in cell-inoculated media. Putative
identifications of the 20 compounds with the highest VIP scores for
the T cell PLS-DA and the 20 compounds with the highest VIP scores
for the CHO PLS-DA combined into one table. KI: Kovats index, calculated
(Calc) and as reported in the literature (Lit); MS Score: Score of
acquired mass spectrum compared to the NIST mass spectral database.

| | | | | | VIP Score | |
|---|---|---|---|---|---|---|
| | KI | KI | | MS | (if > 1) | |
| Compound | (Calc) | (Lit) | CAS # | Score | T cells | CHO |
| 2-pentadecanone | 1696 | 1694 | 2345-28-0 | 77.04 | 1.56 | |
| unknown 12 | 1553 | | | | 1.56 | 1.85 |
| 3,5-bis(1,1-dimethylethyl)- | 1363 | | 125281-21-2 | 81.65 | 1.56 | 1.85 |
| 4-ethyl-1H-pyrazole | | | | | | |
| unknown 13 | 2072 | | | | 1.56 | 1.84 |
| 3,5-dimethoxy-4- | 1497 | 1447 | 5/7/6638 | 64.70 | 1.55 | 1.87 |
| hydroxytoluene | | | | | | |
| unknown 14 (alkylated | 1563 | | | | 1.54 | 1.63 |
| phenol) | | | | | | |
| 3,4-dimethoxybenzoic acid | 1666 | 1670 | 93-07-2 | 70.51 | 1.54 | 1.83 |
| unknown 15 (alcohol) | 1984 | | | | 1.54 | |
| unknown 16 (ketone) | 2018 | | | | 1.54 | |
| unknown 17 | 1345 | | | | 1.54 | 1.85 |
| 3,5-bis(1,1-dimethylethyl)- | 1586 | 1527 | 18712-47-5 | 64.21 | 1.54 | 1.74 |
| 4-methyl-1H-pyrazole | | (est) | | | | |
| unknown 18 | 1858 | | | | 1.54 | |
| unknown 19 (alcohol) | 1786 | | | | 1.54 | |
| 3,5-di-tert-butyl-4- | 1737 | 1774 | 1620-98-0 | 78.24 | 1.54 | |
| hydroxybenzaldehyde | | | | | | |
| 1-amino-2-naphthalenol | 1724 | 1764 | 2834-92-6 | 69.25 | 1.53 | |
| | | (est) | | | | |
| butyl citrate | 2111 | 2150 | 77-94-1 | 97.19 | 1.53 | |
| undecane | 1100 | 1100 | 1120-21-4 | 93.71 | 1.53 | |
| γ-dodecalactone | 1674 | 1673 | 2305-05-7 | 91.82 | 1.53 | 1.84 |
| unknown 20 (fatty acid | 2139 | | | | 1.52 | |
| derivative) | | | | | | |
| unknown 21 (benzene | 1655 | | | | 1.52 | 1.29 |
| dervative) | | | | | | |
| 4-methyl-quinazoline | 1329 | 1363 | 700-46-9 | 87.77 | 1.52 | 1.82 |
| cholesterol | >2400 | 3075 | 57-88-5 | 73.74 | 1.51 | 1.75 |
| 3,5-di-tertbutyl-4- | 1809 | 1903 | 14035-33-7 | 92.94 | 1.51 | 1.84 |
| hydroxyacetophenone | | (est) | | | | |
| unknown 22 (alkylated | 2091 | | | | 1.48 | 1.84 |
| ester) | | | | | | |
| p-benzoquinone | 1459 | 1458 | 719-22-2 | 87.31 | 1.33 | 1.77 |
| sulfurous acid, nonyl 2- | 1345 | | 1000309-12- | | 1.29 | 1.77 |
| propyl ester | | | 0′71.73 | | | |
| 3,5-di-tertbutyl-4- | 1754 | 1774 | 1620-98-0 | 78.24 | 1.26 | 1.85 |
| hydroxybenzaldehyde | | | | | | |
| 5-hexyldihydro-2(3H)- | 1463 | 1463 | 706-14-9 | 94.67 | 1.09 | 1.86 |
| furanone | | | | | | |
| 1-methyl-2(1H)- | 1653 | 1669 | 606-43-9 | 81.69 | | 1.86 |
| quinolinone | | | | | | |
| unknown 23 (alkylated | 1624 | | | | | 1.85 |
| acetophenone) | | | | | | |
| unknown 10 | 1251 | | | | | 1.82 |
| dihydro-5-pentyl-2(3H)- | 1359 | 1360 | 104-61-0 | 89.74 | | 1.79 |
| furanone | | | | | | |
| 7,9-di-tert-butyl-1- | 1911 | 1917 | 82304-66-3 | 96.80 | | 1.77 |
| oxaspiro(4,5)deca-6,9- | | | | | | |
| diene-2,8-dione | | | | | | |
| methyl ether-β-citronellol | 1588 | | 1000333-81-4 | 70.90 | | 1.76 |

Similar to gas exhaust, chemical sensors could be attached to the media waste lines of the bioreactors to monitor target compounds related to cellular health or to perform untargeted analysis to warn users when the waste stream has deviated from a “normal” state. This could help optimize media perfusion rates by monitoring waste and nutrient concentrations within the bioreactor.

### Conclusion

We observed a shift in the specific VOC profile of bioreactor gas exhaust as cell cultures expanded over the course of 8 days. These profiles were used to create PLS regression models that could predict cell culture densities. The volatile compounds most relevant to cell culture expansion for CHO and T cells were putatively identified and discussed. Additionally, measurements of VOCs were made directly in cell-inoculated media during the final day of the experiment. Cell-inoculated media samples were rich in VOCs not present in liquid media controls (no cells present). A PLS-DA analysis revealed the volatile compounds most relevant to the cell cultures and were putatively identified and discussed. Thus, it has been demonstrated that is possible to use VOC-based detection methods on either gas or liquid waste lines of bioreactors to monitor cell health.

Further, by determining a population size and/or density of cells by VOC-based detection, at least one process parameter related to the cell culture process may be controlled. For example, a controller connected to a bioreactor system may be adapted to alter the cell culture parameters in response to the determination of the intensity of VOCs collected and their chemical species and an estimated density or population of cells in the bioreactor based on the determined the intensity of VOCs.

The controller, thus, may adjust chemical and biophysical parameters to further increase expansion, inform harvesting decisions, and control the chemical environment through culture media changes.

Although one embodiment of a cell culture system has been described and illustrated, it will be apparent to the skilled addressee that additions, omissions and modifications are possible to those embodiments without departing from the scope of the invention claimed. For example, the invention has been demonstrated using CHO cell and T cells, however it would be apparent to the skilled addressee that the invention could be employed with equal effect to assess populations of other cells such as, but not exclusively, for therapeutic applications: other lymphocytes such as so-call natural killer cells (NK cells), tumour infiltrating lymphocyte cells (TIL cells); different sub-groups of T cell such as regulatory T cell (Treg cells); antigen-presenting cells such as dendritic cells (D cells); modified cells such as chimeric antigen receptor modified T cells (CAR-T cells), gamma-delta T cells (γδ T cells); and for research, cell populations of other cells such as Vero cells.

1. A method for monitoring cell density during cell expansion resulting from a cell culture process in a bioreactor comprising the steps of:
a) cultivating cells in a bioreactor culture chamber according to a cell culture process having cell culture parameters;
b) during said process, introducing cell culture fluid inputs and generating waste materials;
c) determining the intensity of volatile organic compounds (VOCs) and their chemical species in the waste materials; and
d) estimating the density or population of cells in the bioreactor based on said determination.

2. The method of claim 1, wherein said waste materials include bioreactor headspace gases, and/or filtered liquid or solid waste, and said VOCs include gas phase and/or dissolved or suspended VOCs respectively.

3. The method of claim 1, wherein the waste materials are isolated or removed from the bioreactor chamber prior to said determining.

4. The method of claim 3, wherein said isolation is achieved by an isolation filter allowing only the passage of gases out of the chamber and inhibiting the passage of contaminants into the chamber.

5. The method of claim 1, wherein, during or after said process, the VOCs are collected from said waste materials prior to said determining.

6. The method of claim 5 wherein, said collecting includes exposing the waste materials to a collective element and said determining step includes subjecting the collective element to a chemical detector, for example mass spectrometry (MS) or proton transfer reaction MS, to provide said intensity and profile of VOCs.

7. The method of claim 5, wherein said collecting and said determining are conducted continually, periodically or intermittently.

8. The method of claim 1, wherein said estimating includes assessing the change, and/or rate of change of the VOC concentration/profile.

9. The method of claim 1, wherein said cells are CHO or T cells and the estimation of cell density includes the measurement of the concentration of one or more of alkanes, alkenes, alkynes, carbonyls, esters, alcohols, arenes, acids, amides, amines, carbohydrates, steroids, proteins, nucleic acids and oximes.

10. The method of claim 9, wherein said measurement includes the measurement of the increase in concentration of VOCs, for example, docosane and/or other alkanes.

11. The method of claim 9, wherein a) where said cells are CHO cells, then the measurement includes the measurement of the decrease in concentration of VOCs or b) where said cells are T cells, then the measurement includes the measurement of the decrease in concentration of VOCs, for example, benzaldehyde and/or other aldehydes.

12. The method of claim 8, wherein the ratio of VOCs, for example the ratio of measured alkanes, esters, alcohols and oximes, is used to determine cell density/concentration.

13. The method of claim 1, wherein said estimation is used to control at least one process parameter related to the cell culture process.

14. The method as claimed in claim 13, wherein said estimation is used to alter or enhance said cell culture parameters and/or said cell culture fluid inputs.

15. The method claim 1, further comprising providing an indication of cell viability, health, and/or nutrient utilization based upon the estimated density or population of cells over time.

16. A cell culture system arranged for monitoring cell density during cell expansion resulting from a cell culture process; the system comprising:
a) a bioreactor including a culture chamber suitable for cultivating cells;
b) a controller for conducting a cell culture process according to cell culture parameters;
c) at least one cell culture fluid input and at least one waste materials output;
d) one or more VOCs sensors or collectors present in or at the waste output; and
e) means for determining the intensity of VOCs sensed or collected and their chemical species.

17. The system of claim 16, wherein the controller is configured to estimate the density or population of cells in the bioreactor based on a determined intensity of VOCs.

18. The system of claim 16, wherein said at least one waste materials volume includes: a bioreactor headspace for head space waste gases, and/or a waste gas outlet, and/or an area in the chamber where waste fluids collect, and/or a fluid waste collection line or vessel, and/or a fluid circulation line, and/or an area in the chamber where waste solids collect, and/or a solid waste collection line or vessel, and/or a solid waste circulation line.

19. The system of claim 16, wherein said one or more VOC collectors include a collection element such as sorptive element at least partially within the waste materials volume.

20. The system of claim 16, wherein the system further includes an isolation filter allowing only the passage of gases out of the chamber and inhibiting the passage of contaminants into the chamber, and wherein said waste material volume is downstream of said filter thereby isolating the volume from the chamber.

21. The system of claim 16, wherein means for determining the intensity of VOCs collected and their chemical species is a chemical detector, for example mass spectrometer (MS) or proton transfer reaction MS.

22. The system claim 16, wherein said controller is adapted to control at least one process parameter related to the cell culture process based the determination of the intensity of VOCs collected and their chemical species.

23. The system claimed in claim 22, wherein said controller is adapted to alter the cell culture parameters in response to the determination of the intensity of VOCs collected and their chemical species.

24. The system claim 16, wherein the controller is configured to provide an indication of cell viability, health, and/or nutrient utilization based upon the estimated density or population of cells over time.
